(19) United States
(12) Patent Application Publication
(10) Pub. No.: US 2022/0142632 A1
(43) Pub. Date: May 12, 2022

(54) SPREADER TOOL FOR SEPARATING TISSUES DURING SURGICAL OPERATIONS

(71) Applicant: ALLYON, Saint Jean Bonnefonds (FR)

(72) Inventor: Lionel SIMON, Solaize (FR)

(73) Assignee: ALLYON, Saint Jean Bonnefonds (FR)

(21) Appl. No.: 17/434,439

(22) PCT Filed: Mar. 9, 2020
(86) PCT No.: PCT/FR2020/050475

§ 371 Date: Aug. 27, 2021

(30) Foreign Application Priority Data
Mar. 7, 2019 (FR) 19/02306

(51) Int. Cl. A61B 17/02

(52) U.S. Cl. CPC A61B 17/0206; A61B 2017/00477; A61B 17/0293

(57) ABSTRACT

A spreader tool for separating soft tissues in an outward direction during surgical operations, including a linkage system designed to be arranged in a main surface, and at least two valves having an elongate front part with an exterior side for pressing against the tissues, which is designed to be arranged substantially perpendicular to the main surface, and having a rear face designed to be arranged in the main surface for attaching the linkage system, said linkage system comprising at least one elongate linkage element including a plurality of connecting points designed for connecting the at least two valves to one another or for connecting at least one valve to at least one other elongate linkage means; said tool being notable in that each valve comprises, on its rear face, two pegs designed to fit into holes in the linkage elements.

### TECHNICAL FIELD

The present invention concerns a spreader tool for separating soft tissues during surgical operations.

### STATE OF THE ART

An increasing number of surgeries on the rachis are performed by a posterior approach or an anterior approach or an anterolateral approach, also called retroperitoneal approach, which, in contrast with the posterior approach, allows reducing muscle disinsertions, bleeding and infections, restoring a better lordosis and therefore improving the results by facilitating the recovery of the muscles.

After incision of the tissues at the surface, different systems for separating the soft tissues comprising valves for pressing against these soft tissues, attached on a rigid structure, to clear access to the rachis in order to perform the surgery.

In general, the valves include an elongated shape having on one side, called front portion, a curved shape in the transverse direction. It is used at least two valves linked to one another by their rear ends by means of a mechanical system, which spreads these ends to press the convex external face of their front portions on the tissues in order to separate them and hold them.

A known spreader tool, disclosed in particular by the document US-A1-2005159650, includes a linkage system of at least two valves therebetween, intended to be attached in a plane transverse to the rear end of these valves in order to hold their spacing and their angular inclination with respect to the direction perpendicular to this plane.

The linkage system includes different components allowing forming a closed loop, receiving at least two valves, whose geometry could be adjusted to obtain the desired spacing and inclinations of the front portions of these valves. A clamp, arranged perpendicular to the plane of the linkage system, is maneuvered to apply the spreading stress on the soft tissues completing blocking of the linkage system holding the valves in position.

Another known spreader tool, disclosed in particular by the document US-A1-5931777, includes a clamp arranged in the plane of the linkage system, in order to spread the valves attached at the ends of the jaws of this clamp apart in a perpendicular direction, and curved blocking bars which fix the position of this clamp once spreading is achieved.

Another known spreader tool, disclosed in particular by the document WO-A1-2005016131, includes a clamp disposed in the plane of the linkage system, a threaded rod linking the two grips of the clamp allowing setting the spreading of the jaws of this clamp, and valves attached at the ends of these jaws for holding the spacing of the tissues.

Nonetheless, these linkage systems do not allow carrying out the spreading progressively while adapting to any anatomical variability, and then fixing the surgical field in place, as it has been installed on each particular patient.

Another problem of these spreader tools is that the linkage system generally forms a closed loop completely surrounding the surgical field, which hinders the passage of the hands of the surgeon towards the area to be operated.

In addition, these known spreader tools include linkage systems comprising several complex parts, with geometries that might have cavities that are difficult to clean, generally made of a metallic material, which results in high manufacturing costs. It is possible to reuse them for another surgery, but it is then necessary to provide for a dismounting of all parts in order to carry out a complete sterilization, and then a remounting, which results in an additional occupancy time of the workforce.

Moreover, the complex mechanisms of the linkage system, generally comprising pivots, notches, teeth or racks to achieve the spreading, are prone to wear which requires a periodic control and a replacement of components which are time-consuming, and requiring transport and storage logistics.

Moreover, mention may be made of the document U.S. Pat. No. 1,707,689 which describes a self-retaining spreader device suitable for surgical use. Said device comprises an adjustable quadrilateral frame composed by two slit L-shaped elements each arm of which is provided with a slot, a square-shaped hole being provided at the corner and the internal ends of the slots being enlarged. The device also includes valves with one hook at each end, the rear face of the valves being provided with a slot whose center is enlarged. The two frame elements interlock by means of bolts and wingnuts, the slots in the elements of the frame allow setting them as desired, and these slots, in cooperation with the slots of the valves, allow setting the blades of the frame.

This device type has the drawback of being tricky to set up, handling of the bolts and of the wingnuts being long and difficult during a surgery, and the linkage arms completely close the surgical field, which hinders the passage of the hands of the surgeon to the area to be operated.

### DISCLOSURE OF THE INVENTION

In particular, the present invention aims at avoiding these drawbacks of the prior art.

To this end, and in accordance with the invention, there is provided a spreader tool for separating soft tissues in an outward direction during surgical operations, comprising a linkage system designed to be arranged in a main surface, and at least two valves having an elongate front portion with an exterior side for pressing against the tissues, designed to be arranged substantially perpendicular to the main surface, and having a rear face designed to be arranged in the main surface for attaching the linkage system, said linkage system including at least one elongate linkage element comprising a plurality of connecting points designed for connecting the at least two valves to one another or for connecting at least one valve to at least one other elongate linkage element; said tool being remarkable in that each valve includes, on its rear face, two pegs designed to fit into holes of the linkage elements.

An advantage of this spreader tool is that the elongate linkage elements are simple to link to one another and to the valves, in particular by superimposing them on top of one another, and by rapidly and simply attach them by selecting the positions, to fix the set with determined distances and/or orientations thanks to the connecting points allowing for different positions.

It is possible to use two valves or more, and one, two linkage elements or more, to form the linkage system, with a rapid locking of the position of these valves where appropriate. It should be noted that the linkage elements could form an open contour that clears access to the surgical area. In addition, it is also possible to block the distance and the orientation of the valves in pairs and thus progressively form a tailor-made frame.

Thus, such a spreader tool allows for numerous mounting configurations, so that the surgeon could assemble the valves and the elongate linkage elements according to a geometry that he considers as the most suitable for the surgical act to be completed.

Moreover, the valves and the linkage system having simple shapes, with linkages comprising for example pegs fitted into holes, without any complex mechanism, it is possible to make them easily and economically by molding of a rigid plastic material, or by sheet metal bending, which results in a components that could be discarded after use. In this manner, the manipulations and sanitary risks caused by a reprocessing of components are reduced. In case of reuse, the absence of a complex mechanism facilitates decontamination and/or cleaning and/or reprocessing.

The spreader tool according to the invention may further include one or more of the following features, which may be combined together.

Advantageously, the pegs of the rear faces include a shoulder at their ends, designed to fit into a hole of a linkage element forming a buttonhole, and then to slide into this buttonhole so as to prevent a disengagement. It should be observed that the difference in the diameter between the peg and its shoulder prevents disengagement as soon as the different elements are tensioned.

Advantageously, the linkage elements include a plurality of setting positions, such as for example holes or slots arranged along their length, forming the connecting points therebetween. The choice of these different setting positions left to the surgeon allows controlling both the position and the tensioning of the assembly.

In addition, the linkage elements may include a series of holes which are evenly spaced apart.

In particular, the even spacing may correspond to that of the pegs located on the rear portion of the valves.

In particular, the spreader tool may include flat linkage elements bent in the direction of the main surface.

According to one embodiment, the spreader tool includes two bent linkage elements each comprising on one side of this elbow a series of aligned holes or a rectilinear slot, forming connecting points designed to be superimposed between the two linkage elements.

In particular, the spreader tool may include two movable pegs, or an interference latch comprising two pegs, designed to fit into superimposed holes of the two linkage elements.

Alternatively, each bent linkage element linking two valves, includes a rectilinear slot laterally offset with respect to the axis passing through these valves, comprising blocking notches over at least one side.

In addition, on one linkage element, the notches may form a rack comprising teeth inclined in a direction opposite to the linkage with the valve, receiving a tip of a peg linked to the other linkage element.

In particular, the spreader tool may include two identical linkage elements.

In particular, an edge of the external contour of two linkage elements may include notches forming a rack, the linkage system comprising a ring receiving these two superimposed linkage elements thereinside, having one edge of its internal contour bearing on the notches.

In particular, the rear face of the valves may have a cylindrical surface, formed for each valve by two arms extending along the transverse direction of the cylinder, which slip on top of one another while reversing this superimposition on both sides.

Advantageously, the valves and the linkage system are made by injection of a plastic material.

### BRIEF DESCRIPTION OF THE DRAWINGS

The invention will be better understood and other features and advantages will appear more clearly upon reading the description hereinafter provided as example, with reference to the appended drawings wherein:

FIG. 1 is a perspective view of a spreader tool with two valves according to the invention, comprising a linkage system with two curved bars forming a closed contour;

FIG. 2 is a perspective view of a variant of a spreader tool with two valves, with two curved bars forming an open contour;

FIG. 3 is a perspective view of a variant of a spreader tool with three valves, with two curved bars forming an open contour;

FIG. 4 is a perspective view of a variant of a spreader tool with two valves and a wire, with three curved bars forming a closed contour;

FIG. 5 is a perspective view of a variant of a spreader tool with three valves, with two curved bars and one straight bar forming a closed contour;

FIG. 6 is a perspective view of a variant of a spreader tool with three valves, with one curved bar and two straight bars forming a closed contour;

FIG. 7 is an exploded perspective view of a variant of a spreader tool with two valves, with two square-like shaped bars forming an open contour, linked by two pegs inserted into buttonholes;

FIG. 8 is a perspective view of a variant of a spreader tool with two valves, with two square-like shaped bars linked by a latch combining the function of the two pegs;

FIG. 9 is an exploded perspective view of a variant of a spreader tool with two valves, with two square-like shaped bars linked by a latch forming a system sliding in one direction and self-blocking in the other;

FIG. 10 is a top view of the square-like shaped bars linked by a latch of the variant of the spreader tool with two valves according to the invention represented in FIG. 9;

FIG. 11 is a perspective view of a variant of a square-like shaped bar of a spreader tool according to the invention, comprising a fixed peg arranged outside a groove;

FIG. 12 is a top view of two square-like shaped bars linked by a latch of the variant of the spreader tool with two valves according to the invention represented in FIG. 11;

FIG. 13 is a perspective view of a variant of a square-like shaped bar of a spreader tool according to the invention, comprising an offset peg;

FIG. 14 is a perspective view of a variant of a bar of a spreader tool according to the invention, comprising a rack;

FIG. 15 is a top view of two square-like shaped bars of the variant of the spreader tool with two valves according to the invention represented in FIG. 14;

FIG. 16 is a top and perspective view of a variant of a spreader tool according to the invention, comprising a ring linking the racks of each bar;

FIG. 17 is a perspective view of a variant of a spreader tool according to the invention, comprising a linkage system with one single bent bar;

FIG. 18 is an exploded perspective view of a variant of a spreader tool according to the invention comprising a valve and an arcuate bar according to an arcuate main surface;

FIG. 19 is a perspective view of the variant of a spreader tool according to the invention represented in FIG. 18;

FIG. 20 is a perspective view of a clamp for the variant of the spreader tool according to the invention represented in FIGS. 18 and 19;

FIG. 21 is a perspective view of the clamp between the valves of the spreader tool according to the invention represented in FIGS. 18 and 19;

FIG. 22 is a perspective view of the clamp in the spread position of the valves of the spreader tool according to the invention represented in FIGS. 18 and 19.

### EMBODIMENT OF THE INVENTION

In the following description of the spreader tool according to the invention, the same reference numerals refer to the same elements. The different views are not necessarily plotted to scale.

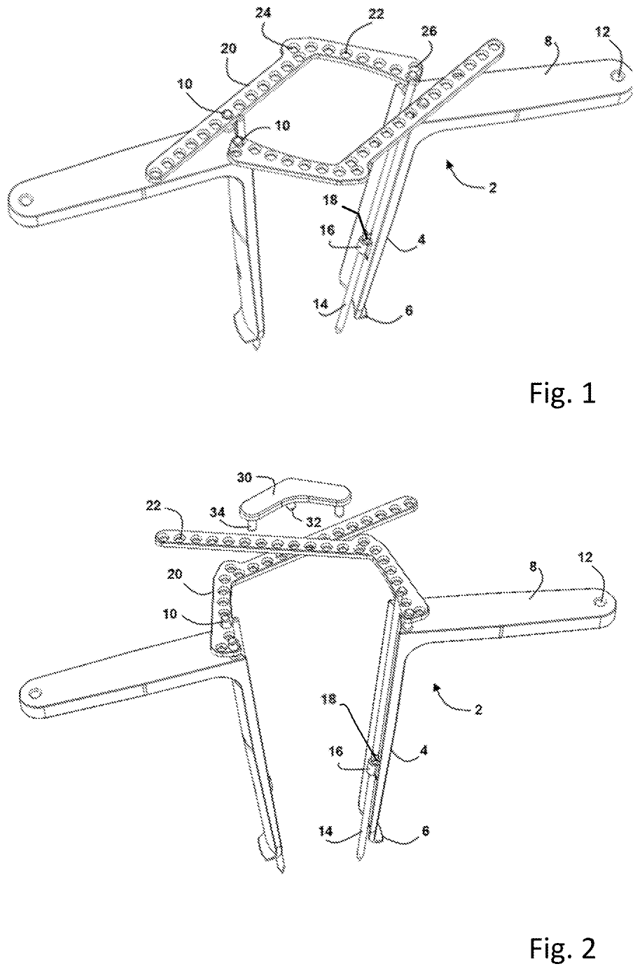

FIG. 1 shows a spreader tool comprising two identical valves 2 arranged opposite one another, whose upper ends forming an elongate rear transverse face 8 arranged in a main plane forming a main surface, are linked together by a linkage system comprising two bent bars 20 forming a closed contour arranged on this plane. Next, the interior and exterior sides are in the main plane or surface, defined as being respectively at the center and outwards of the linkage system.

Each valve 2 includes an elongate front portion 4 having a substantially constant cross-section, comprising a curved shape whose exterior convex side is intended to press against the tissues.

The front end of the elongate portion 4 includes a small flange 6 directed outwardly according to an angle of about 105° more or less 20°, enabling hooking on the tissues to avoid a longitudinal slipping of this elongate portion or a slipping of the separated tissues beneath the valve 2. Thus, said flange 6 allows scraping the soft tissues of the surface of the vertebrae, for example, and preventing the aorta artery and the vena cava from passing beneath the valve 2.

On each valve 2, the lower end of the elongate rear face 8 includes two pegs 10 aligned perpendicularly to the longitudinal direction of this face, the exterior end comprising a hole 12 enabling hooking of the rear face.

The linkage system includes two bent bars 20, each bar forming a linkage element having on each side of this elbow a large side and a short side, forming a flat surface on the rear face 8 of the valves 2, comprising a series of holes 22 evenly spaced along the bar according to a standard step. Each bar 20 includes outwardly of the elbow a first additional hole 24, and at the end of the short side outwardly of this elbow a second additional hole 26. These additional holes enable an angular variation between the valve 2 and the bar(s) 20.

A wire 14 having a pointy front end, is fitted into a guide 16 forming a loop arranged in the axis of the concave side of the front portion 4 of the valve 2, so as to be able to slide along this front portion in order to press with this tip in a bone, such as for example a vertebra, thereby holding the lower portion of the valve 2 in position. Each wire 14 includes a top 18 bearing on the upper guide edge 16 when the tip of the wire 14 is inserted into the bone thereby avoiding an excessively deep insertion of the wire 14 into the bone and also thereby ensuring anchorage of the valve 2 on the bone, preventing the latter from rising and letting the aorta artery and the vena cava pass.

Each bent bar 20 receives in the end hole of the short side a peg 10 of a valve 2, and at the middle of the large side a peg of the other valve. The two bars 20 are placed opposite one another so as to form the closed contour.

After having fastened the tips of the wires 14 on bones, a separation of the upper portions of the valves 2 is performed thereby displacing the soft tissues backwards until the surgeon obtains enough space for the operation. Afterwards, he performs an engagement of the pegs 10 into the holes 22 of the bars 20 corresponding to the spacing in a simple and rapid way, to hold the position of the valves with respect to one another.

It should be noted that the valves 2 and the bars 20 have simple shapes, easy to obtain by molding of a rigid plastic material. The bent flat bars 10 have a large width, confer a good rigidity on the elbow formed by these bars, which allows obtaining the stiffness easily with the plastic material.

FIG. 2 has two bent bars 20, each receiving in the first and third holes 22 of the short side a peg 10 of a valve 2. In this manner, an angular wedging of each bar 20 on the rear face 8 of each valve 2 is achieved.

A flat blocking part 30 forming an elbow widthwise includes on its lower face a central blocking peg 32 and at each end an end blocking peg 34. The distance between the central peg 32 and each end peg 34 corresponds to a distance between holes 22 of the bars 20.

After having obtained the desired spacing of the valves 2, the surgeon inserts the central peg 32 of the blocking part 30 into two superimposed holes of the two bars 20 and an end peg 34 into another hole of each bar, which fix the angular positioning of these two bars with respect to one another in a simple and rapid way.

It is obtained a linkage system having an open contour, this opening completely clearing access for the passage of the hand of the surgeon during the surgical operation.

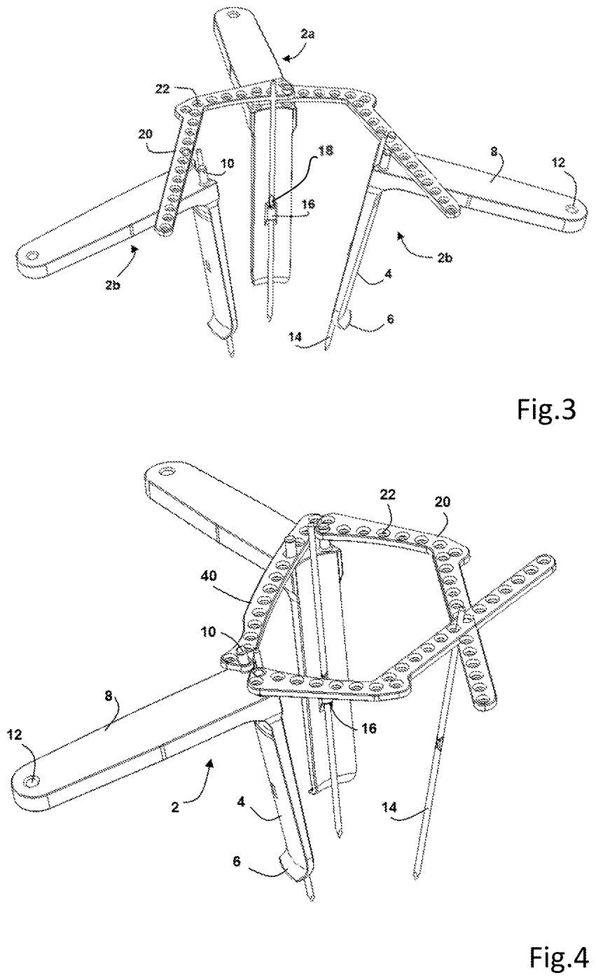

FIG. 3 shows the ends of the short sides of the two superimposed bent bars 20, so as to engage the two pegs 10 of the rear face 8 of a central valve 2a into the first and third end hole 22 of each short side. In this manner, an angular wedging of the two bars 20 with respect to this central valve 2a is achieved.

A peg 10 of a lateral valve 2b is inserted into a hole 22 at the middle of the large side of each bent bar 20, which allows fixing a spacing between the three valves. A linkage system having an open contour linking three valves 2 is also obtained.

FIG. 4 shows a substantially straight bar 40 forming a linkage element, comprising a series of holes 22 distributed along its length according to the same standard step, receiving in two holes two pegs 10 of two valves 2 so as to hold spacing thereof. For this purpose, the two pegs 10 of the two valves 2 are used which are brought close to one another. The two elongate rear faces 8 of the valves 2 form an angle of about 120°.

Each remaining peg 10 of each valve 2 is inserted into the end hole of the short side of a bent bar 20. The rear end of a wire 14 is inserted into two superimposed holes 22 at the middle of the large side of the two bent bars 20, to close the contour of the linkage system thereby positioning the two valves 2 and this wire.

FIG. 5 shows a similar linkage system with the straight bar 40 linking two valves 2, receiving instead of the wire 14 inserted into the two superimposed holes of the two bent bars 20, a valve 2c comprising its two pegs 10 each inserted into one hole of each bar.

In the same manner, a closed contour rigidly holding three valves 2 is obtained.

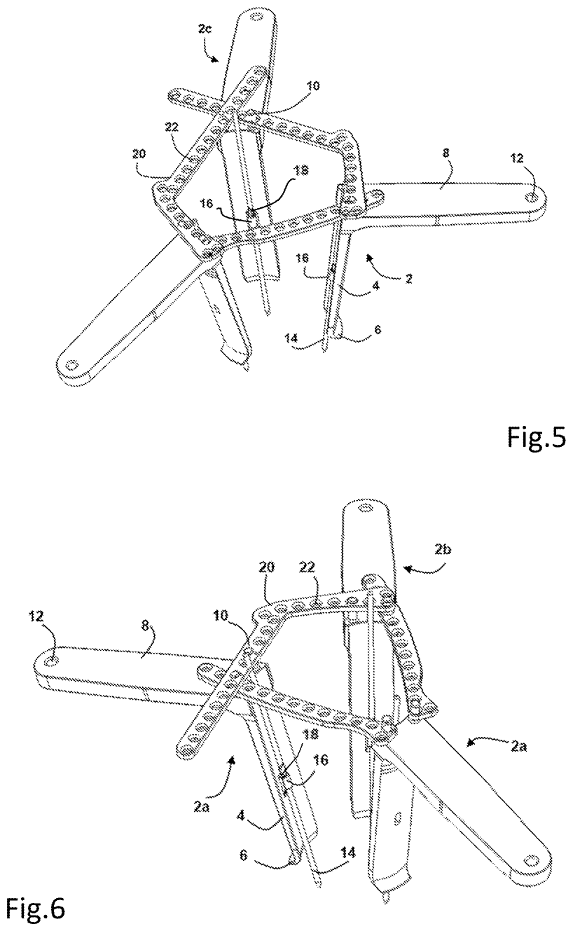

FIG. 6 shows a central valve 2a each peg 10 of which is inserted into an end hole of a straight bar 40, the two pegs 10 of two lateral valves 2b being fitted into a hole arranged close to the other end of these two straight bars.

A bent bar 20 links the two lateral valves 2b by fitting on the two pegs 10 of each of these valves, so as to obtain a closed contour of the linkage system.

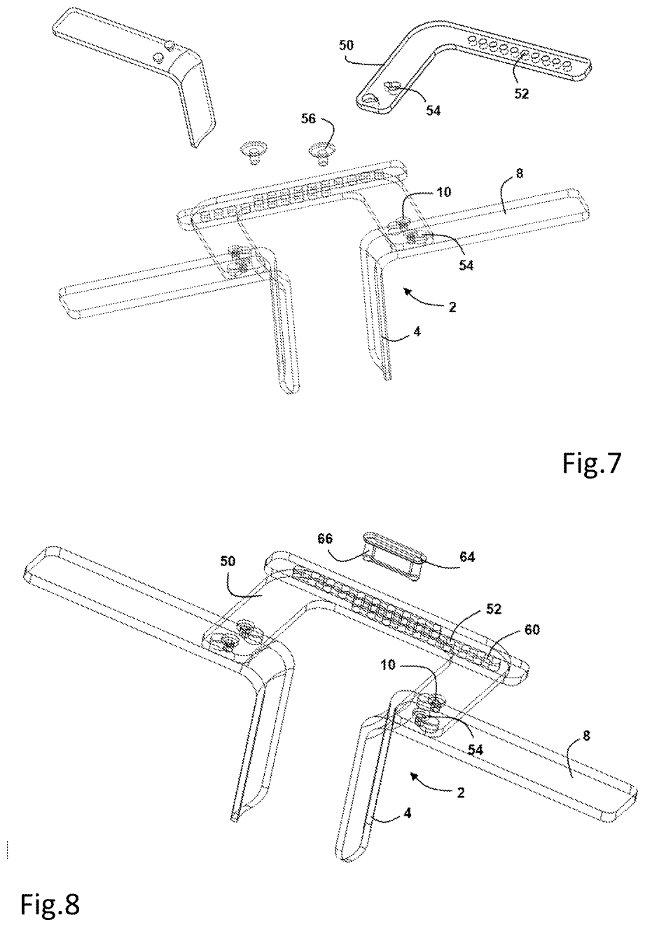

FIG. 7 shows an elongate rear face 8 of each valve 2 including on the interior side two pegs 10 aligned transversely, each comprising a small-diameter cylindrical body terminating in a flat head with a larger diameter.

Each square-like shaped bar 50 at substantially 90°, forming a linkage element, includes over a large side a series of evenly spaced holes 52, and at the end of the short side two holes each forming a buttonhole 54. It should be noted that, by “substantially 90°”, it should be understood an angle of 90° more or less 10° . Moreover, the branches of the square-like shaped bar 50 may form an angle comprised between 60° and 120° and each branch may have any shape, for example non-rectilinear, yet without departing from the scope of the invention.

Each buttonhole 54 includes a small-diameter hole fitted to the body of the peg 10, these two small holes being aligned along the longitudinal axis of the short side. Each buttonhole extends in the transverse direction of the short side, outwardly of the elbow for the end buttonhole and inwardly for the other buttonhole, by a large-diameter hole letting the head of the peg 10 pass.

After having engaged the two pegs 10 of each valve 2 in the two buttonholes 54 of a square-like shaped bar 50, a rotational movement of the bar setting its short side perpendicular to the elongate rear face 8, engages the bodies of the pegs in to the small-diameter holes which hooks the bars beneath the head of the pegs.

Afterwards, the valves 2 are spread apart to achieve the desired separation of the tissues, by superimposing holes 52 of the bars 50. Then, movable pegs 56 comprising a rear head are inserted into two superimposed holes 52, thereby achieving a complete positioning of the two bars 50 with respect to one another and holding the spacing of the valves 2.

FIG. 8 shows two square-like shaped bars 50 similar to the bars shown in FIG. 7, comprising on each bar, instead of the row of holes 52, a longitudinal slot 60 receiving by superimposition a series of evenly spaced circular holes 52, centered on the axis of this slot, having a diameter slightly larger than the slot.

The two movable pegs 56 are replaced with an interference latch 64 achieving the same function, comprising two parallel cylindrical portions 66 forming the two pegs fitting in two circular holes 52, linked by a longitudinal bar fitting into the slot 60, and a rear head with a larger width.

In the same manner, the interference latch 64 is inserted after having superimposed the circular holes 52 of the two bent bars 50, which holds the spacing of the valves 2.

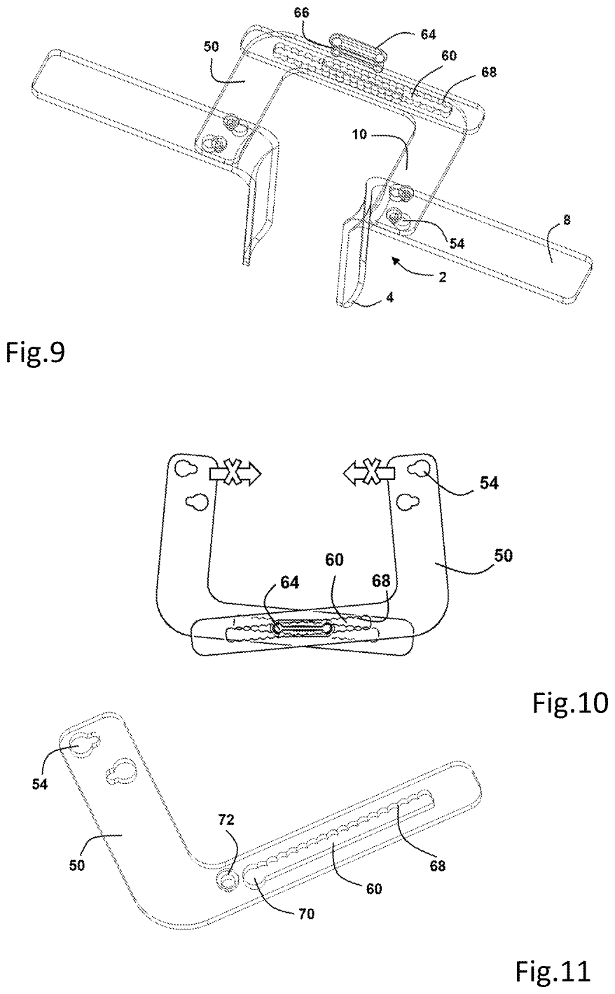

FIG. 9 shows two square-like shaped bars 50 similar to the bars shown in FIG. 8, comprising for the slot 60 a close succession of circular holes which overlap each other sufficiently to form an elongate passage with notches 68 on each side, enabling a free sliding of the cylindrical portions 66 of the interference latch 66 along this length when the holes of the two bars are superimposed.

In this manner, the interference latch 64 is fitted into the elongate passes of the two bars 50, and then the two valves are spread apart while keeping these two passages aligned with one another, the latch sliding freely to follow this movement.

FIG. 10 shows the free spreading of the two valves 2 which superimposes the two large sides with their slots 60, and then after having obtained the sufficient spacing, the release of the bars 50 which causes, with the push of these valves, an inclination of the two slots with respect to one another as shown in this figure, by pressing the two cylindrical portions 66 of the interference latch 64 to the bottom of the notches 68.

By the force applied on the valves 2, a self-blocking of the two bars 50 with respect to one another is achieved which increases with this force, while keeping the distance between these valves.

FIG. 11 shows two square-like shaped bars 50 similar to the bars shown in FIG. 9, comprising on the side of the slot 60 arranged inside the elbow a close succession of circular holes forming notches 68, the exterior side of the elbow remaining smooth.

A shouldered peg 72 provided with a large head is fixed in the axis of the slot 60, between its end and the elbow of the bar 50. The end of the slot 60 arranged on the elbow side, has a large-diameter hole forming a buttonhole 70 enabling the passage of the head of the shouldered peg 72.

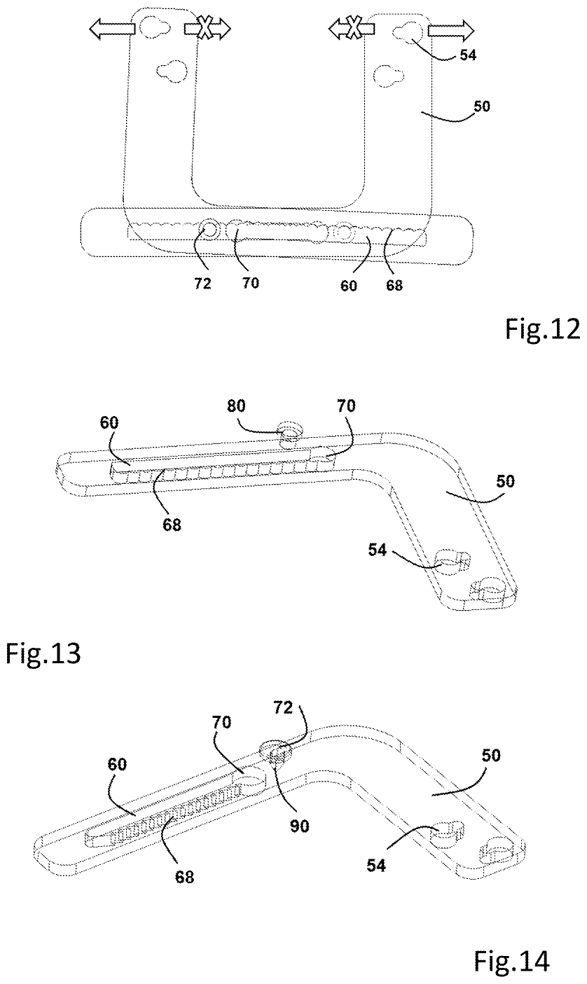

FIG. 12 shows, after having engaged each shouldered peg 72 in a buttonhole 70 of the other bar 50, a free sliding of these pegs in the slots 60 by spreading the valves 2 apart and by holding the two slots superimposed as shown in this figure.

It should be noted that the smooth side of the slot 60 enables a spreading of the valves 2 which presses the shouldered pegs 72 on these smooth sides, without disturbing the sliding, in contrast with the slots shown in FIG. 9 which require holding the alignment of these slots to obtain the spacing.

In the same manner, upon release of the bars 50, the valves 2 apply a force which tends to incline the two slots 60 with respect to one another, while pressing on the cylindrical portion of each shouldered peg 72 in the notches 68 of the other bar.

FIG. 13 shows a square-like shaped bar 50 similar to the bar shown in FIG. 11, including a peg 80 offset with respect to the axis of the slot 60 which replaces the attached shouldered peg 72.

The set-up of the linkage system is similar after having inserted the two detached pegs 80 into the slots 60 of the two bars 50, with an unblocking in the direction of spreading of the valves 2 and a blocking in the other direction.

FIG. 14 show a square-like shaped bar 50 similar to the bar shown in FIG. 11, with the notches 68 on the side of the slot 60 internal to the elbow forming a rack comprising teeth inclined outwards of the large side of this bar. The central portion of the shouldered peg 72 has a tip 90 directed on the same side as the notches 68.

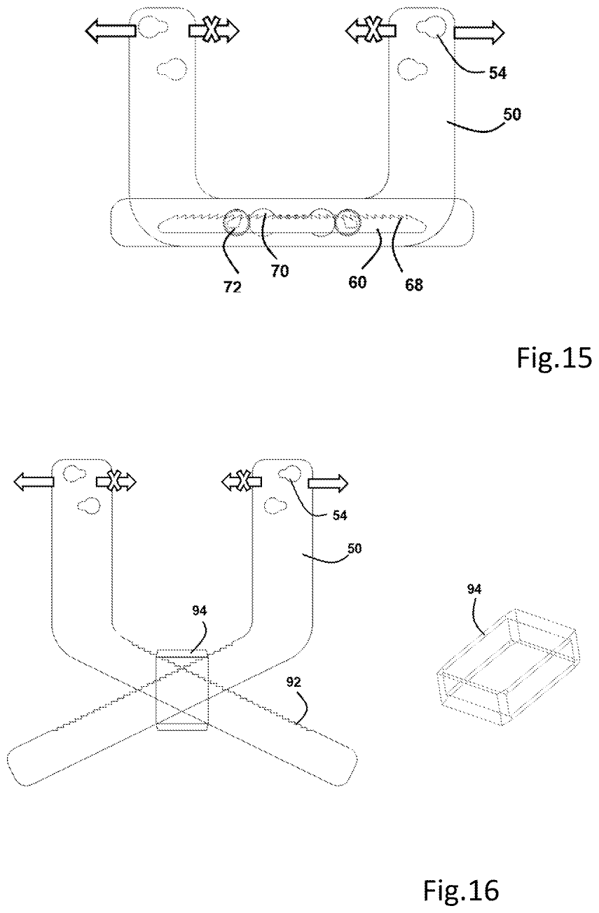

FIG. 15 shows, in the direction of spreading of the valves 2, the shouldered pin 72 bearing on the smooth side of the slot 60, which enables a free sliding. The tightening force of the two valves 2 causes a fitting of the tip 90 into a notch 68 of the rack, which prevents the movement.

FIG. 16 shows bars 50 including an elbow by about 120°, the long portion of each bar being provided on the edge internal to the elbow with a rack 92 with teeth inclined towards the end of this long portion.

A linkage ring 94 has a flat internal contour allowing receiving the two superimposed long portions of the two bars 50.

The width of the internal contour is slightly larger than the width of the long portions of the bars 50, which allows holding the short portions parallel, the long portions forming an angle of about 30° therebetween.

When the valves 2 tend to get close to one another, the blocking is achieved by the notches of the racks 92 which stick on the edges of the internal contour of the linkage ring 94 directed towards these valves. Conversely, when spreading the valves 2 apart, the smooth sides opposite to the racks 92 bear on the internal contour of the linkage ring 94, the racks no longer bearing which enables sliding.

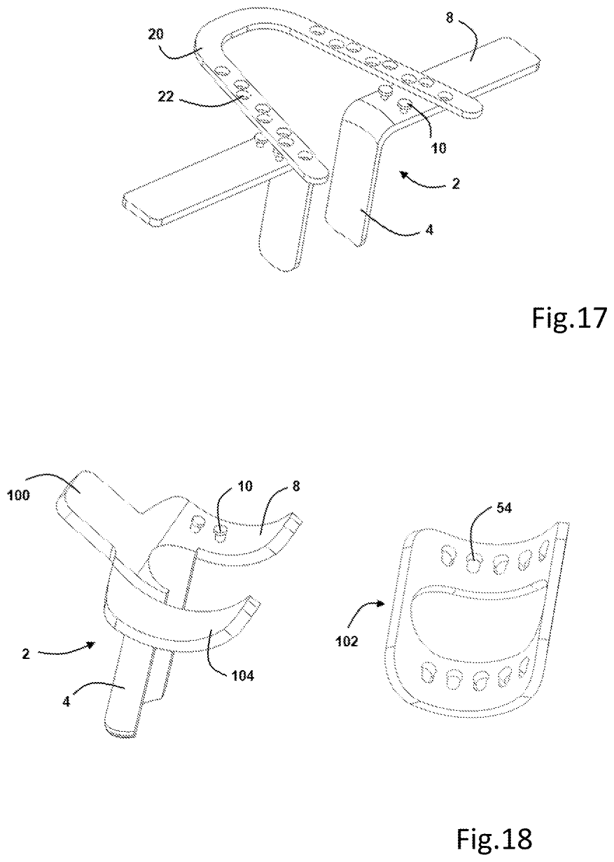

FIG. 17 shows a curved bar 20 with circular holes 22, comprising two straight ends forming an angle of about 30° therebetween, forming a passage therebetween which opens outwardly.

Each of the two valves 2 includes an elongate rear face 8 comprising two pegs 10 aligned transversely, provided with a shoulder at their ends, which fit into two holes 22 of one of the straight ends of the bar 20.

After having placed and spread the valves 2 apart sufficiently, the holes 22 of the bar 20 corresponding to this spacing are selected, by adapting the distance with respect to the central curvature ensuring the proper spacing of the valves. The shoulder of each peg 10 coming on top of the bar 20 after having crossed the circular hole 22, slips sidewise by the effect of the transverse forces applied on the vales 2 which blocks the linkage by preventing a removal of the peg.

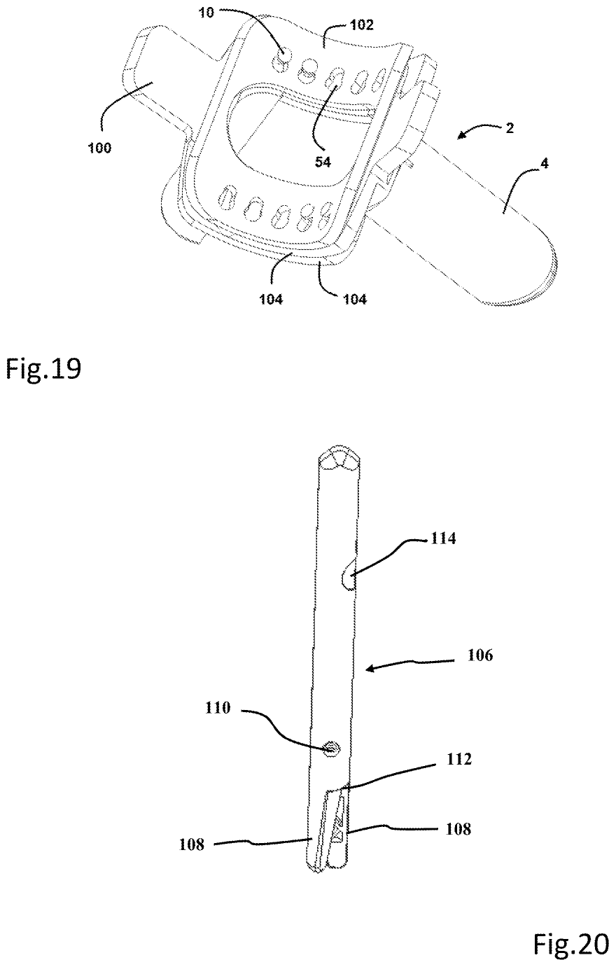

FIGS. 18 and 19 show two identical valves 2, each comprising a rear face 8 which is curved according to a cylindrical surface, including two parallel arms 104 which extend according to a transverse direction of the cylinder.

The two arms 104 have concave upper faces whose heights are slightly offset, the height offset being substantially equal to the thickness of said arms 104, and the curvatures of the concave upper faces of said arms 104 have slightly different radii, the radius of curvature of the concave upper face of the first arm 104 extending beneath the first arm 104 being substantially equal to the radius of curvature of the convex lower face of the second arm 104 which extends above the first arm 104, so as to adjust an arm of one of them slipping beneath that of the other one, with a role reversal on both sides. A guidance of the valves 2 relative to one another is ensured, with a pivot connection about the axis of the cylindrical shape.

Each rear face 8 extends outwardly by a handle 100 disposed according to a transverse direction of the cylinder, allowing making the valves 2 pivot easily to spread their front portions 4 apart. Said handles 100 also allow blocking the position of the valves 2 by means of a table arm that is not represented in the figures.

The arm 104 of each valve 4 having the smallest diameter of curvature, includes two pegs with a shoulder 10 aligned according to the transverse direction of the cylinder.

A curved plate 102 forming a bar, constitutes a loop having two parallel straight sides each fitting on an upper arm 104 of the valves 2. Each straight side includes buttonholes 54 aligned along the length of this side, which extend in the transverse direction.

After having set the spacing of the valves 2 by acting on the handles 100, the plate 102 is lowered by inserting the shoulders of the pegs 10 into the large portion of the corresponding buttonholes 54, and then this plate is slipped transversely to lock the assembly of the pegs.

It should be noted that this variant allows promoting a spreading at the level of the surgical site, that is to say at the valve end 2, while avoiding an excessively large incision of the soft tissues that cover the surgical site, such as muscles and skin.

Moreover, referring to FIGS. 20 to 22, in order to easily and rapidly obtain a spreading of said valves, the device according to the invention also includes a clamp 106 constituted by to semi-cylindrical arms 108 hinged about an axis 110 in the proximity of the proximal end of the arms 108, the proximal end of the arms 108 being slender at a height corresponding to the height slightly below that of the valve 2 to form a stop 112. The distal end of the arms 108, opposite to the slender proximal end, is slightly pointy and, by being directed downwards, the clamp being in the closed position, allows penetrating the soft tissues and scraping the bone. In addition, the arms 108 include above the axis 110, recesses 114 allowing for an easy gripping of the arms 108 to make them pivot about the axis 110.

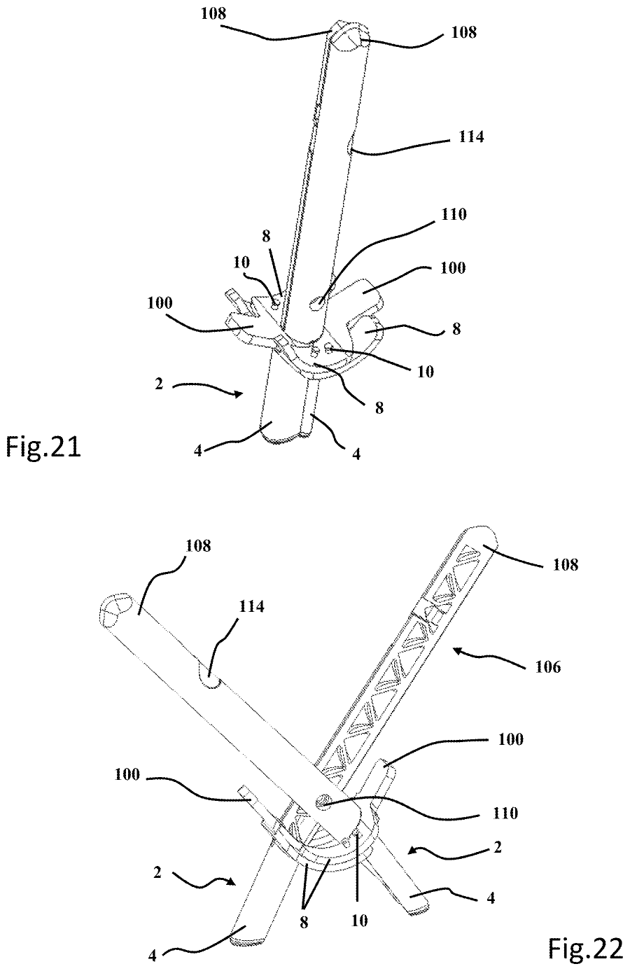

Thus, referring to FIG. 21, the valves 2 are inserted into the soft tissues after incision thereof, the lower portions of the valves 2 extending in parallel. Afterwards, the clamp 106 in the closed position is inserted between the valves 2 until the pointy distal ends bear on the bone to scrap it. The clamp 106 is removed and then returned, still in the closed position, and inserted between the valves 2 until the stops 112 bear on the rear faces 8 of the valves 2. It should be observed that the position of the stops 112 allows having a common axis of rotation between the clamp 106 and the cylindrical surface of the two valves 2. Then, referring to FIG. 22, the arms 108 of the clamp 106 are pivoted about the axis 110 to open the latter and impart the pivoting and, ultimately, the spreading of the valves 2. Said valves 2 are then held in this spread position by means of the handles 100, either manually or by means of a table arm that is not represented in the figures, and then the clamp 106 is removed and the plate 102 is placed as described before.

In general, the valves 2 as well as the different linkage systems are simple and economical elements, free of any complex mechanism, which could be easily made by molding of a rigid plastic material. Systems that are disposable easy to configure in any case and which provide the surgeon with a wide field of action are obtained

In general, the pegs and the holes receiving them, to fix the bars 20, 40, 50 on the valves 2, may be arranged in the reverse order by mounting these pegs on the bars and the holes on the valves.

1. A spreader tool for separating soft tissues in an outward direction during surgical operations, comprising a linkage system designed to be arranged in a main surface, and at least two valves having an elongate front portion with an exterior side for pressing against the tissues, designed to be arranged substantially perpendicular to the main surface, and having a rear face designed to be arranged in the main surface for attaching the linkage system, said linkage system including at least one elongate linkage element comprising a plurality of connecting points designed for connecting the at least two valves to one another or for connecting at least one valve to at least one other elongate linkage element, wherein each valve includes, on its rear face, two pegs designed to fit into holes of the linkage elements.

2. The spreader tool according to claim 1, wherein the pegs of the rear faces include a shoulder at their ends, designed to fit into a hole of a linkage element forming a buttonhole, and then to slide into this buttonhole so as to prevent a disengagement.

3. The spreader tool according to claim 1, wherein each linkage element includes slots arranged along their length, forming the connecting points therebetween.

4. The spreader tool according to claim 1, wherein the linkage elements include a series of holes which are evenly spaced apart.

5. The spreader tool according to claim 3, wherein it includes flat linkage elements bent in the direction of the main surface.

6. The spreader tool according to claim 5, wherein it includes two bent linkage elements each comprising on one side of this elbow a series of aligned holes or a rectilinear slot, forming connecting points designed to be superimposed between the two linkage elements.

7. The spreader tool according to claim 6, wherein it includes two movable pegs, or an interference latch comprising two pegs, designed to fit into superimposed holes of the two linkage elements.

8. The spreader tool according to claim 6, wherein each bent linkage element linking two valves, includes a rectilinear slot laterally offset with respect to the axis passing through these valves valves, comprising blocking notches over at least one side.

9. The spreader tool according to claim 8, wherein on one linkage element, the notches form a rack comprising teeth inclined in a direction opposite to the linkage with the valve, receiving a tip of a peg linked to the other linkage element.

10. The spreader tool according to claim 5, wherein it includes two identical linkage elements.

11. The spreader tool according to claim 1, wherein an edge of the external contour of two linkage elements includes notches forming a rack, the linkage system comprising a ring receiving these two superimposed linkage elements thereinside, having one edge of its internal contour bearing on the notches.

12. The spreader tool according to claim 1, wherein the rear face of the valves has a surface curved according to a cylindrical surface, formed for each valve by two arms extending along the transverse direction of the cylinder, which slip on top of one another while reversing this superimposition on both sides.

13. The spreader tool according to claim 1, wherein the valves and the linkage system are made by injection of a plastic material.
